(19) United States
(12) Patent Application Publication
(10) Pub. No.: US 2023/0102599 A1
(43) Pub. Date: Mar. 30, 2023

(54) HIGHLY RESISTANT STARCH AND MANUFACTURING PROCESS THEREFOR

(71) Applicant: LODAAT, LLC, Oak Brook, IL (US)

(72) Inventor: Rajiv KHATAU, Oak Brook, IL (US)

(21) Appl. No.: 17/904,933

(22) PCT Filed: Feb. 26, 2021
(86) PCT No.: PCT/US2021/019834

§ 371 Date: Aug. 24, 2022

Related U.S. Application Data
(60) Provisional application No. 62/981,736, filed on Feb. 26, 2020.

(51) Int. Cl. A61K 31/718; C08B 30/04; A61K 36/81; A23L 29/212; A23L 33/21; A23L 19/12; A23L 3/46

(52) U.S. Cl. CPC A61K 31/718; C08B 30/048; A61K 36/81; A23L 29/212; A23L 33/21; A23L 19/12; A23L 3/46; A61K 2236/51; A61K 2236/30; A61K 2236/13; A23V 2002/00

(57) ABSTRACT

The present disclosure relates to highly resistant starch compositions, methods for preparing such highly resistant starch compositions, and methods of using such highly resistant starch compositions to treat or prevent a disease or condition associated with dysregulation and/or dysfunction of the intestinal microbiome.

### CROSS REFERENCE TO RELATED APPLICATIONS

This application claims priority to U.S. Provisional Patent Application No. 62/981,736, filed on Feb. 26, 2020, the entire contents of which are herein incorporated by reference.

### FIELD OF THE INVENTION

The present disclosure provides highly resistant starch compositions, methods for manufacturing the same, and products, such as food products or beverages, containing such compositions.

### BACKGROUND OF THE INVENTION

A healthy community of metabolically active microbes in the intestinal microbiome is essential for host (e.g., human) health. In particular, short-chain fatty acids (SCFAs), which are molecules produced by intestinal microbes by fermenting dietary components, are essential for host health. SCFAs, and, in particular, butyric acid and/or its derivatives, contribute to host health by facilitating epithelial integrity, regulating inflammation, and influencing gene expression in host colonocytes.

Intestinal microbes responsible for SCFA production are fueled by complex polysaccharides derived primarily from dietary components. Resistant starch is a polysaccharide that resists digestion in the small intestine and ferments in the large intestine and, thus, provides the fuel for intestinal microbes as well as a substrate for SCFA production. Type 1 resistant starch (RS1) is found in grains, seeds, and legumes and resists digestion because it is bound within the fibrous cell walls. Type 2 resistant starch (RS2) is inaccessible to human digestive enzymes due to the conformation of the starch itself. RS2 is found in, for example, green bananas and potatoes.

Approximately 90% of the U.S. population does not consume enough dietary fiber to provide SCFA production to maintain promotion and maintenance of a healthy intestinal environment. A typical commercially available resistant potato starch only contains about 50% resistant potato starch by weight.

There is an unmet need for highly resistant starch compositions and products containing such compositions, particularly for promotion and maintenance of a healthy intestinal environment.

### SUMMARY OF THE INVENTION

The present disclosure relates to a highly resistant starch composition and, particularly, a highly resistant potato starch composition. In certain embodiments, the highly resistant starch composition comprises at least 48%, alternatively at least 50%, alternatively at least 55%, alternatively at least 60%, alternatively at least 65%, or alternatively at least 70%, by weight of resistant starch (type 2). In certain embodiments, the highly resistant starch composition comprises from about 60% to about 85% or, alternatively, from about 65% to about 80%, by weight of resistant starch.

The present disclosure also relates to a method for manufacturing a highly resistant starch composition. The method comprises the steps of (a) providing an edible tuber; (b) cleaning the edible tuber; (c) extracting starch, particularly RS2, from the edible tuber; (d) drying the starch to a moisture content of 20% or less; and (e) removing impurities from the starch. In certain embodiments, step (d) comprises air drying, convective drying, and/or spray drying the starch.

The present disclosure also relates to an orally consumable product comprising a highly resistant starch composition disclosed herein and/or a highly resistant starch composition manufactured by a method disclosed herein. In certain embodiments, the consumable product is a food product, a beverage, or a mix product intended to be combined with a food or beverage. In some such embodiments, the food product is a bar, a cookie, a muffin, or the like. In some such embodiments, the beverage is a ready-to-drink (RTD) beverage such as a juice, a shake, or the like. In some such embodiments, the mix product is in the form of a tablet, pellet, powder, granule, or the like which can be added to a food or beverage.

The present disclosure relates to a method for promoting short chain fatty acid production in an intestine of a subject in need thereof. The method comprises orally administering to a subject a consumable product comprising a highly resistant starch composition disclosed herein and/or a highly resistant starch composition manufactured by a method disclosed herein. In certain embodiments, the short-chain fatty acid is butyric acid or a derivative thereof. In some such embodiments, the short-chain fatty acid is butyrate.

The present disclosure also relates to a method for treating or preventing a disease or condition associated with intestinal dysregulation and/or dysfunction, including dysregulation and/or dysfunction of the intestinal microbiome. The method comprises orally administering to a subject a consumable product comprising a highly resistant starch composition disclosed herein and/or a highly resistant starch composition manufactured by a method disclosed herein. In certain embodiments, the disease or condition is selected from the group consisting of graft-versus-host disease, autoimmune diseases, allergies, inflammatory conditions, metabolic disorders, cancers such as colorectal cancer, diabetes, and obesity.

The compositions, methods for manufacturing the compositions, products comprising the compositions, and methods for using the compositions to promote intestinal health are further described herein.

These and other objects of the invention are described in the following paragraphs. These objects should not be deemed to narrow the scope of the invention.

### BRIEF DESCRIPTION OF THE DRAWINGS

For a better understanding of the invention, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art.

FIG. 1 shows flow diagram depicting an exemplary manufacturing process for a highly resistant starch composition.

FIG. 2 shows another flow diagram depicting an exemplary manufacturing process for a highly resistant starch composition.

### DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

This disclosure provides highly resistant starch compositions and methods for manufacturing such highly resistant starch compositions. The highly resistant starch compositions disclosed herein provide a superior solution for decreasing inflammation, regulating satiety, and promoting healthy levels of glucose and insulin. Moreover, the highly resistant starch compositions disclosed herein deliver such benefits with fewer calories, greater convenience, and more efficacy than current commercially available options.

Section headings as used in this section and the entire disclosure are not intended to be limiting.

### A. DEFINITIONS

As used in the specification and the appended claims, unless specified to the contrary, the following terms have the meaning indicated:

The term “about” as used herein means approximately, and in most cases within 10% of the stated value.

The term “butyric acid” (also known under the systematic name butanoic acid), as used herein, refers to a carboxylic acid with the structural formula CH₃CH₂CH₂COOH. The term “butyric acid derivatives” as used herein includes compounds derived from butyric acid, such as salts and esters of butyric acid, which are known as butyrates or butanoates. Non-limiting examples of butyrate salts include sodium butyrate, calcium butyrate, magnesium butyrate, manganese butyrate, cobalt butyrate, barium butyrate, lithium butyrate, zinc butyrate, potassium butyrate, ferrous butyrate, and the like. Non-limiting examples of butyric acid esters include cellulose acetate butyrate, methyl butyrate, ethyl butyrate, butyl butyrate, pentyl butyrate, and the like.

An “effective amount” of a composition, as used herein, includes an amount sufficient to promote microbial production of metabolic products (including, without limitation, butyric acid and derivatives thereof), which can be detected in in a sample, such as a fecal sample, from the subject at specific periods after administration.

The term “prebiotic” refers to an agent that increases the number and/or activity of one or more desired bacteria. A prebiotic may be an agent that allows specific changes both in the composition and/or activity in the gastrointestinal microbiota that may (or may not) confer benefits upon the host. In some embodiments, a prebiotic can be a comestible food or beverage or ingredient thereof.

The term “short-chain fatty acid” or “SCFA” as used herein refers to a carboxylic acid attached to an aliphatic tail (chain) having up to six carbon atoms. Exemplary short-chain fatty acids include but are not limited to formic acid, acetic acid, propionic acid, butyric acid, and valeric acid. Short-chain fatty acids are produced when dietary fibers are fermented in the intestine of a mammals.

A “therapeutically effective amount” refers to an amount effective, at dosages and for periods of time necessary, to achieve the desired therapeutic result, such as treatment or prevention of a disease or condition associated with intestinal dysregulation and/or dysfunction. A therapeutically effective amount of a composition may vary according to factors such as the disease state, age, sex, and weight of the subject, and the ability of the composition to elicit a desired response in the subject.

The terms “treat”, “treating” and “treatment” as used herein refer to a method of alleviating or abrogating a condition, disorder, or disease and/or the attendant symptoms thereof.

### B. METHODS FOR MANUFACTURING HIGHLY RESISTANT STARCH COMPOSITIONS

In at least one aspect, this disclosure is directed to a method for manufacturing a highly resistant starch composition.

In certain embodiments, the method comprises providing a raw material. In some such embodiments, the raw material is a source of resistant starch type 2 (RS2). In certain embodiments, the raw material is an edible tuber. In some such embodiments, the edible tuber is Solanum tuberosum. In some such embodiments, the edible tuber is grown in the northern hemisphere (e.g., northern Europe).

In certain embodiments, the method comprises cleaning the RS2 source. In some such embodiments, a separator is used to remove dirt, stones, and/or other debris from the RS2 source. In some such embodiments, the RS2 source is washed (e.g., with water) to remove dirt, stones, and/or other debris from the RS2 source.

In certain embodiments, the method comprises extracting starch from the RS2 source. In some such embodiments, the RS2 source is crushed and a centrisieve is used to extract starch. Exemplary centrisieves include those available from Larsson® and are often integrated in a system, for example a starch extraction system.

In certain embodiments, the method comprises drying the extracted starch. In some such embodiments, the extracted starch is dried by air drying. In some such embodiments, the extracted starch is dried by convective drying. In some such embodiments, the extracted starch is dried by spray drying. In some such embodiments, a spray drying apparatus such as a cyclone separator may be employed.

In some such embodiments, the inlet air temperature in the spray drying process is from about 100° C. to about 150° C., such as about 110° C., about 120° C., about 130° C., or about 140 ° C. In some such embodiments, the extracted starch is dried by sequential drying processes such as an air drying step or a convective drying step followed by a spray drying step. In some such embodiments, the first drying step and the second drying step provide a partially dried starch having a moisture content of 20% or less.

In certain embodiments, the method comprises removing impurities from the dried starch. In some such embodiments, the dried starch is sieved. In some such embodiments, the dried starch is sieved through a mesh screen. For example, the mesh screen may be a 0.3 mm mesh. In some such embodiments, the dried starch is passed through a separator, such as a magnetic separator. An exemplary magnetic separator has a high magnetic performance, such as about 13200 Gauss.

In certain embodiments, the method comprises the steps of: (a) providing a batch of raw material (e.g., edible tubers); (b) picking out desirable tubers (quality control); (c) cleaning the tubers; (d) drying starch obtained from the tubers; (e) spray-drying the starch obtained from the tubers; (f) sifting out impurities; (g) inspecting and packaging the starch; and/or (h) storing the starch.

### C. HIGHLY RESISTANT STARCH COMPOSITION

In at least one aspect, this disclosure is directed to a highly resistant starch composition. The disclosed highly resistant starch compositions may be prepared by any suitable method, including those described herein.

In certain embodiments, the highly resistant starch composition is derived from an edible tuber. In some such embodiments, the edible tuber is Solanum tuberosum. In some such embodiments, the edible tuber is grown in the northern hemisphere (e.g., northern Europe).

In certain embodiments, the highly resistant starch composition comprises at least 48%, alternatively at least 50%, alternatively at least 55%, alternatively at least 60%, alternatively at least 65%, or alternatively at least 70%, by weight of resistant starch (type 2). In certain embodiments, the highly resistant starch composition comprises from about 60% to about 85% or, alternatively, from about 65% to about 80%, by weight of resistant starch (type 2). In some such embodiments, the highly resistant starch composition comprises about 50%, about 55%, about 60%, about 65%, about 70%, about 75%, about 80%, or about 85% by weight of resistant starch (type 2).

Determination of resistant starch (type 2) content in a composition can take place according to methods known in the art. For example, a sample of the composition may be first digested for a period of time (e.g., from 4 to 16 hours) with human digestive enzymes (e.g., pancreatic alpha-amylase, protease, and/or amyloglucosidase) to remove digestible starch and protein. Subsequently, the methods described in McCleary, et al, Measurement of Resistant Starch by Enzymic Digestion in Starch and Selected Plant Materials—Collaborative Study. J. AOAC Int., 85, 1103-1111 (2002) and/or McCleary, et al., Measurement of available carbohydrates, digestible, and resistant starch in food ingredients and products. Cereal Chemistry, 97, 114-137 (2019) as well as modified enzymatic-gravimetric assays such as described in AOAC Methods 2002.02 or 2009.01, each of which are herein incorporated by reference in their entirety, may be employed to determine resistant starch content.

In certain embodiments, the highly resistant starch composition comprises dietary fiber (e.g., as determined according to AOAC Method 991.43 for determining total, soluble, and insoluble dietary fiber in foods). In some such embodiments, the highly resistant starch composition comprises total dietary fiber and/or soluble dietary fiber. In some such embodiments, the highly resistant starch composition comprises at least 0.5% by weight of total dietary fiber. In some such embodiments, the highly resistant starch composition comprises from about 0.5% to about 2.0% by weight of total dietary fiber. In some such embodiments, the highly resistant starch composition comprises about 0.5%, about 1.0%, about 1.5% or about 2.0% by weight of total dietary fiber. In some such embodiments, the highly resistant starch composition comprises at least 0.5% by weight of soluble dietary fiber. In some such embodiments, the highly resistant starch composition comprises from about 0.5% to about 2.0% by weight of soluble dietary fiber. In some such embodiments, the highly resistant starch composition comprises about 0.5%, about 1.0%, about 1.5% or about 2.0% by weight of soluble dietary fiber.

In certain embodiments, the highly resistant starch composition, upon ingestion by a subject, promotes production of short-chain fatty acids, such as butyric acid and/or butyric acid derivatives (e.g., butyrates).

### D. PRODUCTS CONTAINING HIGHLY RESISTANT STARCH COMPOSITIONS

In at least one aspect, this disclosure is directed to a product comprising a highly resistant starch composition disclosed herein and/or a highly resistant starch composition manufactured by a method disclosed herein. In certain embodiments, the product is provided as discrete units such as tablets, capsules, pouches, or sachets, each containing a predetermined amount of the highly resistant starch composition.

In certain embodiments, the product is powder. In some such embodiments, the product is a fine white powder. The powder may be packaged in bulk or in individual units for consumption (e.g., in pouches or sachets). In certain embodiments, the powder is intended to be mixed with a food product or beverage (e.g., as a drink preparation). The food product or beverage may be intended for human or animal consumption.

In certain embodiments, the highly resistant starch composition is incorporated into a food or beverage and the product is a prepared food item or a ready-to-drink beverage.

In certain embodiments, the product is a prepared food item. Exemplary prepared food items include, but are not limited to, bars, biscuits, cheese, chews, chocolate, cookies, dips, gummies, ice cream, pudding, sauces, and soups. In some such embodiments, the prepared food item does not require cooking after admixture with the highly resistant starch composition.

In certain embodiments, the product is an RTD beverage. Exemplary RTD beverages include, but are not limited to, dairy beverages, juices, and tea beverages.

In certain embodiments, the product is a drink preparation, such as a powder or granule. The powder or granule can be mixed with any suitable liquid for ingestion such as water, coffee, and the like.

In certain embodiments, the product is an animal feed or an animal feed supplement.

Highly resistant starch compositions, as described herein, can be formulated as a nutraceutical composition, such as a medical food, a nutritional or dietary supplement, a food or beverage product, and include a nutraceutically acceptable carrier. As used herein, a “nutraceutically acceptable carrier” refers to, and includes, any and all solvents, dispersion media, coatings, antibacterial and antifungal agents, isotonic and absorption delaying agents, and the like that are physiologically compatible. In some embodiments, the nutraceutically acceptable carrier is suitable for pediatric use.

In certain embodiments, the nutraceutical composition is orally ingestible and in the form of a powder or granule (optionally contained within a pouch or sachet), tablet, or capsule.

Highly resistant starch compositions, as described herein, can be formulated as a pharmaceutical composition and include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. In some embodiments, the pharmaceutically acceptable carrier is suitable for pediatric use.

In certain embodiments, the pharmaceutical composition is an oral dosage form, preferably a solid oral dosage form (e.g., a tablet). In some such embodiments, the solid oral dosage form may comprise pharmaceutically acceptable excipients such as excipients that function as binders, glidants, lubricants, and fillers. Thus, a solid oral dosage form comprising a highly resistant starch composition further optionally comprises one or more conventional pharmaceutically acceptable excipients.

Exemplary amount or dose of the highly resistant starch (RS2) in the composition include at least 1 gram (g), alternatively, at least 5, alternatively, at least 10 g, alternatively, at least 15 g, alternatively, at least 20 g, alternatively, at least 25 g, or, alternatively, at least 30 g. In certain embodiments, the amount of RS2 in the composition is from about 1 g to about 200 g, alternatively, from about 10 g to about 200 g, alternatively, from about 20 g to about 150 g, alternatively, from about 30 g to about 100 g, or, alternatively, from about 40 g to about 80 g. In certain embodiments, the amount of RS2 in the composition is about 1 g, about 5 g, about 10 g, about 20 g, about 30 g, about 40 g, about 50 g, about 60 g, about 70 g, about 80 g, about 90 g, or about 100 g.

### E. METHODS OF USE

In at least one aspect, the present disclosure includes a method for promoting short-chain fatty acid production in an intestine of a subject in need thereof. In certain embodiments, the short-chain fatty acid comprises acetic acid, acetic acid derivatives, propionic acid, propionic acid derivatives, butyric acid, butyric acid derivatives, and combinations thereof. In certain embodiments, the short-chain fatty acid comprises butyric acid and/or butyric acid derivatives (e.g., butyrates).

The method comprises administering to a subject in need thereof an effective amount of a highly resistant starch composition disclosed herein and/or a highly resistant starch composition manufactured by a method disclosed herein. In certain embodiments, the composition is administered orally.

Without wishing to be bound by theory, it is believed that the resistant starch is metabolized or degraded and then fermented by microbes in the large intestine to a variety of products, including short-chain fatty acids, such as butyric acid and derivatives thereof.

Degrading bacteria may comprise, for example, Bifidobacterium species and Ruminococcus bromii.

SCFA-producing bacteria may comprise Clostridium cluster XIVa and/or Clostridium cluster IV, such as Faecalibacterium prausnitzii. In particular, butyrate-producing species of bacteria include but are not limited to Butyrivibrio fibrisolvens, Clostridium butyricum, Clostridium chartatabidum, Clostridium kluyveri, Clostridium pasteurianum, Eubacterium limosum, Eubacterium rectale, Faecalibacterium prausnitzii, Fusobacterium nucleatum, and Ruminococcus bromii. Particularly preferred species include Clostridium chartatabidum, Eubacterium rectale, and Ruminococcus bromii.

Production of fermentation products, including short-chain fatty acids such as butyric acid and/or butyric acid derivatives, can be performed by methods known in the art. In certain embodiments, a fecal sample collected from the subject can be assessed to determine the presence and/or quantity of fermentation products. For example, a fecal sample may be processed by sequential membrane filtration and subsequently assayed to measure acetic, propionic, and/or butyric acid in the filtrate.

In an exemplary embodiment, quantification of short-chain fatty acids is performed by high-performance liquid chromatography (HPLC). In some such embodiments, an HPLC system is equipped with a dual UV (214 nm for short-chain fatty acids/refractive index detector (for ethanol). In some such embodiments, the mobile phase is 0.01 N H₂SO₄ at a flow rate of 0.6 ml/min with the column temperature at 50° C., and an injection volume of 10 μl. External standards (e.g., 0.1-20 mM for acetic, propionic, and butyric acids) can be used to generate a standard curve.

Reduced concentrations of butyrate have been associated with a variety of diseases and conditions, including graft-versus-host disease (GvHD), kwashiorkor (protein malnutrition), colon cancer, and obesity.

Benefits of butyrate include, but are not limited to, decreased inflammation, regulation of satiety, promotion of healthy blood glucose and/or insulin levels, promotion of epithelial barrier.

In certain embodiments, the highly resistant starch composition is derived from an edible tuber. In some such embodiments, the edible tuber is Solanum tuberosum. In some such embodiments, the edible tuber is grown in the northern hemisphere (e.g., northern Europe).

Fecal short-chain fatty acid concentrations are responsive to dietary supplementation with resistant potato starch (RPS). In particular, ssupplementation with RPS increased butyrate concentrations by an average of 29% (p<0.001) and acetate by an average of 21% (p=0.0012). See Baxter et al., Dynamics of human gut microbiota and short-chain fatty acids in response to dietary interventions with three fermentable fibers. mBio 10:e02566-18 (2019).

In at least one aspect, the present disclosure includes a method for treating or preventing a disease or condition associated with intestinal dysfunction (including, but not limited, to abnormal or abnormally functioning microbes in the gastrointestinal tract) in a subject in need of such treatment or prevention. In certain embodiments, the disease or condition is selected from the group consisting of graft-versus-host disease, autoimmune diseases, allergies, inflammatory conditions, gastrointestinal disorders, kwashiorkor (protein malnutrition), metabolic disorders, cancers such as colorectal cancer, diabetes, and obesity. Exemplary autoimmune diseases include, but are not limited to, systemic lupus, idiopathic thrombocytopenic purpura, Sjogren's syndrome, haemolytic uremic syndrome, and scleroderma. Exemplary inflammatory conditions include, but are not limited to, rheumatoid arthritis and systemic lupus erythematosus. Exemplary gastrointestinal disorders include, but are not limited to, irritable bowel syndrome (IBS), intestinal inflammation, functional bowel disease, and inflammatory bowel disease including Crohn's disease and ulcerative colitis.

The method comprises administering to a subject in need thereof a therapeutically effective amount of a highly resistant starch composition disclosed herein and/or a highly resistant starch composition manufactured by a method disclosed herein. In certain embodiments, the composition is administered orally.

Conventional pharmaceutical or nutraceutical practice may be employed to provide suitable formulations or compositions to administer the highly resistant starch composition, as described herein, to a subject.

Exemplary total daily doses of resistant starch (RS2) include at least 1 gram (g)/day, alternatively, at least 5 g/day, alternatively, at least 10 g/day, alternatively, at least 15 g/day, alternatively, at least 20 g/day, alternatively, at least 25 g/day, or, alternatively, at least 30 g/day. In certain embodiments, the total daily dose of RS2 is from about 1 g/day to about 100 g/day, alternatively, from about 10 g/day to about 80 g/day, alternatively, from about 15 g/day to about 75 g/day, or, alternatively, from about 20 g/day to about 70 g/day. Compositions containing RS2 may be administered in amount to provide such total daily doses. For example, to achieve a total daily dose of resistant starch (RS2) from about 28 g/day to about 34 g/day, a subject may be administered from about 40 g/day to about 48 g/day of a composition containing about 70% by weight resistant starch (RS2).

The resistant starch, whether in the form of a composition, food product, RTD beverage, may be administered to the subject in a single dose per day or in multiple doses per day such as, for example, 2 times per day, 3 times per day, or 4 times per day.

Factors affecting the preferred dosage regimen include the type, age, weight, sex, diet, and condition of the subject, the desired effect to be achieved, and/or the severity of any disease or condition desired to be treated or prevented. Thus, the dosage regimen actually employed can vary widely, and therefore, can derive from the preferred dosage regimen set forth above.

### F. EXAMPLES

The manufacturing methods and highly resistant starch compositions described herein will be better understood by reference to the following examples, which are included as an illustration of and not a limitation upon the scope of the invention.

### Example 1: Exemplary Manufacturing Process for Highly Resistant Starch Composition

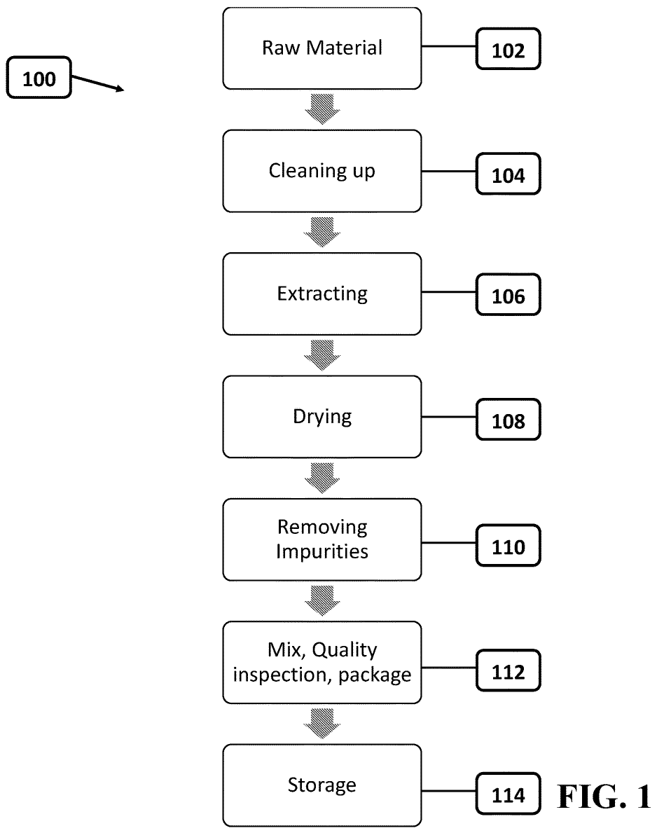

FIG. 1 depicts an exemplary method 100 for manufacturing a highly resistant starch composition. In step 102, a raw material (e.g., edible tuber) is provided. In step 104, the raw material is cleaned. For example, the raw material can be cleaned using a separator and/or by washing with water. In step 106, starch is extracted from the raw material. In step 108, the extracted starch is dried (e.g., by air drying, convective drying, and/or spray drying) to a moisture content of 20% or less. In step 110, impuries such as metal are removed from the dried starch. In step 112, purified dried starch is inspected and packaged. In step 114, the packaged purified dried starch is stored.

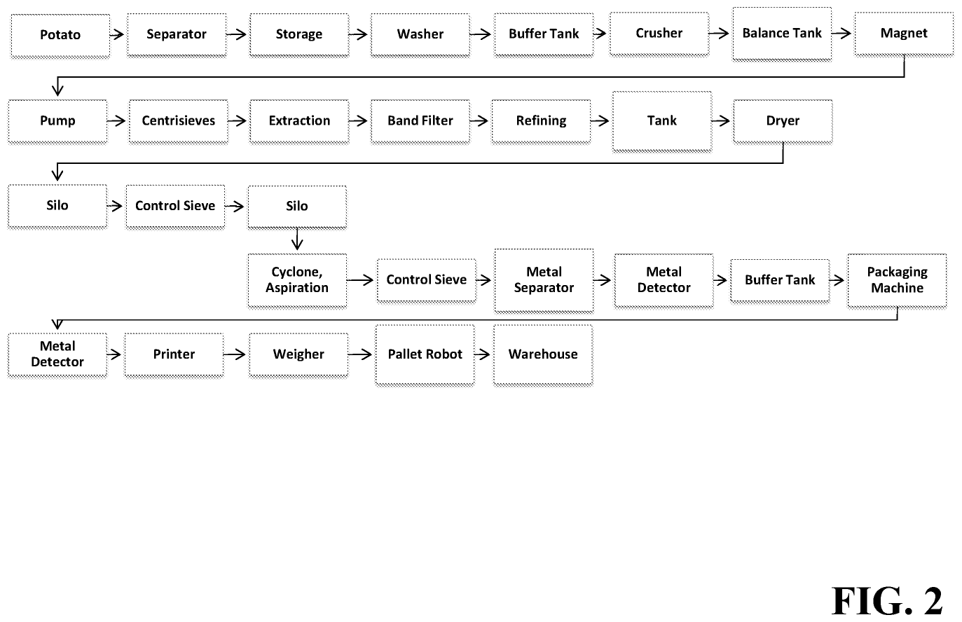

FIG. 2 depicts another exemplary method for manufacturing a highly resistant starch composition beginning with the raw material (e.g., a potato). After quality control, a separator may be used to separate stones, dirt, and/or other debris from selected potatoes. Subusequently, such potatoes may be stored. Following such optional storage, potatoes may be placed in transporation water for transport to a washer. The washer may be used to further remove stones, dirt, and/or other debris. Following washing, the potatoes may be transferred to a buffer tank. Following an optional holding period in the buffer tank, a crusher is used to crush the potatoes. Following crushing, the material may be transferred to a balance tank. Following an optional period in the balance tank, a magnet (e.g., about 13,000 Gauss) may be used to extract metal fragments form the material. Starch may be extracted from the crushed material by centrifugal sieving, such as by using a centrisieve. The pulp can be discarded or moved to a fibre production workflow. Similarly, the juice can be discarded or moved to a protein production workflow. The extracted starch may be subjected to a band filter to remove excess moisture. The extracted starch may be further dried in a dryer (e.g., about 160° C.) to a moisture content of 20% or less. The extracted, dried strach may be transferred to a silo and/or sieved. The extracted, dried strach may be further subjected to a spray drying apparatus such as a cyclone separator. Following spray-drying, the starch may be further sieved (e.g., using a 0.3 mm sieve mesh). The sieved starch may then be passed through a metal separator and/or a metal detector.

### Example 2: Prebiotic Resistant Potato Starch

A fine white powder flour was obtained by processing an edible tuber (Solanum tuberosum) as described herein. The resistance level was determined to be 65%-80% (mean 72.3%).

Product parameters, including physical characteristics and microbiological and pathogen analysis, are provided in Table A.

TABLE A

| | | |
|---|---|---|
| Parameter | Specification | Reference |

Physical Characteristics

| | | |
|---|---|---|
| Description | White fine powder with characteristic | Organoleptic |
| | odor and smell | |
| Sieve test | ≥99.9% pass 100 Mesh | As per USP <786> |
| Moisture | ≤20% | Internal |

Other Characteristics

| Spot | ≤3 | unit/cm2 | As per USP <231> |
|---|---|---|---|
| Protein | ≤0.10% | | AOAC |
| Ash | ≤0.30% | | AOAC |
| Viscosity (4%) 700 cmg | ≥1500 | BU | Internal |
| Electric Conductivity | ≤100 | μ/cm | AOAC |
| SO2 (%) | ≤10.0 | mg/kg | AOAC |
| pH in suspension | 5.0-8.0 | | Internal |
| PSL | PPSL-001 | | Negative |
| Bulk Density | >0.5 | g/ml | USP |

Microbiological and pathogen Analysis

| | | |
|---|---|---|
| Standard Plate Count | NMT <10000 cfu/gm | As per USP <61> |
| Yeast & Molds | NMT <100 cfu/gm | As per USP <61> |
| Escherichia coli | Absent | As per USP <61> |
| Salmonella | Absent | As per USP <61> |
| Pseudomonas aeruginosa | Absent | As per USP <61> |
| Staphylococcus aureus | Absent | As per USP <61> |

Nutrition value is provided in Table B.

TABLE B

| | |
|---|---|
| Energy value (100 g) | 1348 kJ |
| Energy value (100 g) | 317 Kcal |
| Fat | 0.1 g |
| Saturated fat | 0.02 g |
| Carbohydrate | 79 g |
| Sugars | 0 g |
| Protein | 0.1 g |
| Salt | 0.10 g |

### Example 3: Comparative Analysis

A comparative analysis was conducted (Certified Labs LLC, Melville, N.Y.) to determine the amount of resistant starch in a composition prepared as disclosed herein relative to a commercially available potato starch product (Bob's Red Mill® Potato Starch; Milwaukie, Oreg.).

TABLE C

| | Comparative | Test | |
|---|---|---|---|
| Analyte | Sample | Sample | Method Reference |
| Resistant Starch | 57.45% | 61.54% | |
| Total Dietary Fiber | 0% | 0.9% | AOAC 991.43 |
| Soluble Dietary Fiber | 0% | 0.9% | AOAC 991.43 |

The highly resistant starch compositions disclosed herein contain more resistant starch (RS2) as compared to a commercially available potato starch product. In addition, highly resistant starch compositions disclosed herein contain total dietary fiber and soluble dietary fiber whereas the commercially available potato starch product does not.

It will be readily apparent to those skilled in the art that other suitable modifications and adaptations of the compositions and methods of the invention described herein may be made using suitable equivalents without departing from the scope of the invention or the embodiments disclosed herein.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. A process for manufacturing a digestive-resistant starch composition, the process comprising the steps of:
(a) providing an edible tuber;
(b) cleaning the edible tuber;
(c) extracting starch from the edible tuber;
(d) drying the starch to a moisture content of 20% or less; and
(e) removing impurities from the starch.

2. The process of claim 1, wherein step (d) comprises spray-drying the starch.

3. The process of claim 1, wherein step (d) comprises convection drying and subsequently spray-drying the starch.

4. The process of claim 1, wherein the digestive-resistant starch composition comprises at least 60%, preferably at least 70%, resistance starch (type 2) by weight.

5. The process of claim 1, wherein the digestive-resistant starch composition stimulates short-chain fatty acid production upon administration to a subject.

6. The process of claim 5, wherein the short-chain fatty acid is butyric acid or a derivative thereof.

7. The process of claim 1, wherein the edible tuber is Solanum tuberosum.

8. A digestive-resistant starch composition produced by the process of claim 1.

9. A food product comprising the digestive-resistant starch composition of claim 8.

10. A beverage comprising the digestive-resistant starch composition of claim 8.

11. A dosage form comprising the digestive-resistant starch composition of claim 8.

12. The dosage form of claim 11, wherein the dosage form is capsule or a tablet.

13. A method for promoting short-chain fatty acid production in an intestine of a subject, the method comprising administering to the subject the digestive-resistant starch composition of claim 8.

14. The method of claim 13, wherein the short-chain fatty acid is butyric acid or a derivative thereof.

15. The process of claim 1, wherein step (c) comprises centrifugal sieving.

16. The process of claim 15, wherein step (d) comprises convection drying.

17. The process of claim 1, wherein the edible tuber is solanum tuberosum.

18. The process of claim 4, wherein the digestive-resistant starch composition further comprises dietary fiber.

19. The process of claim 4, wherein the digestive-resistant starch composition further comprises from about 0.5% to about 2.0% by weight total dietary fiber.

20. The process of claim 4, wherein the digestive-resistant starch composition further comprises from about 0.5% to about 2.0% by weight soluble dietary fiber.
